(19) United States
(12) Patent Application Publication
(10) Pub. No.: US 2010/0187810 A1
(43) Pub. Date: Jul. 29, 2010

(54) ARRANGEMENT FOR CONNECTING TUBE ELEMENTS IN A VENTILATION DUCT SYSTEM

(76) Inventors: Pontus Andersson, Angelholm (SE); Lars-Ake Mattsson, Bastad (SE); Sten Högman, Grevie (SE)

Correspondence Address: TAROLLI, SUNDHEIM, COVELL & TUMMINO L.L.P., 1300 EAST NINTH STREET, SUITE 1700, CLEVELAND, OH, 44114, US

(21) Appl. No.: 12/439,799

(22) PCT Filed: Sep. 7, 2007
(86) PCT No.: PCT/SE07/00778

§ 371 Date: Mar. 3, 2009

Related U.S. Application Data
(60) Provisional application No. 60/843,208, filed on Sep. 8, 2006.

(30) Foreign Application Priority Data
Sep. 8, 2006 (SE) 0601856-8

(51) Int. Cl. A61M 39/10

(52) U.S. Cl. 285/134.1

(57) ABSTRACT

In an arrangement for connecting tube elements of a ventilation duct system, the inner tube (3) has an abutment (7′) for engagement with a matching abutment (8′) of the outer tube (1). By insertion of the inner tube (3) into the outer tube (1), the abutments (7′, 8′) are brought into engagement in a plane (P) perpendicular to the direction of insertion for providing coupling between the abutments and thereby connection of the tubes (1, 3). The abutment (7′) of the inner tube (3) is continuous along the circumference of the tube (3) and is provided at a distance (d) from a free end (20) of the inner tube (3).

### TECHNICAL FIELD

The present invention relates to the field of connecting tube elements and similar components in ventilation duct systems.

### BACKGROUND ART

During the recent decades, designers of ventilation duct systems have tried to find effective methods of connecting tube elements and similar components included in such systems.

In a widely used method, an inner tube is inserted into an outer tube and fastening means (screws or rivets) are driven through the overlapping tube wall section. Normally, the tubes are helically-wound lock-seam tubes of sheet metal. Often, the installer or fitter has to drill holes through the tube walls for the fastening means. This tube connection is cumbersome, time-consuming and requires special tools, as electric drills, screw drivers, riveting machines, etc. Furthermore, the fastening means project into the duct which may disturb the air flow and also cause air leakage, undesired accumulation of dust, etc. There is also a risk that the projections into the duct interfere with and damage cleaning equipment, such as brushes, which is pushed through the duct.

In other tube connecting methods, the tube ends are provided with end beads which are disposed end-to-end and clamped by means of special external clamping devices (see for instance U.S. Pat. No. 6,030,005 and WO-A-05/001323). In certain applications, these methods work well but a disadvantage is that the fitter has to handle separate parts, such as clamping devices, coupling rings, etc.

Still another known tube connecting method is known from CH-A-496,927 which, however, also requires a separate and structurally complicated coupling member between the tube ends.

EP-A-797,038 discloses a tube connecting method where both tube walls are provided with a plurality of interlocking cuts which are cut through the material. The close hook-like cuts have the disadvantage of weakening the tube walls. The intermittent cuts constitute kerfs which may cause undesired longitudinal slots in the thin tube wall. It is also hard to provide precise cuts in the thin tube wall for establishing the locking effect. Furthermore, it is difficult to bring the cuts into matching positions since the tubes have to be precisely orientated. In practice, the cut portions do not flex back after insertion which means that the aimed-at engagement by the cuts is not achieved. Another drawback is that the risk of air leakage at the cuts is relatively high.

A similar prior-art tube connection is known from the Swedish patent application SE 0402011-1 which discloses an outer tube provided with hook-like cuts cut through the tube wall. The cuts are intended to engage shoulders on the outside of an inner tube inserted in the outer tube. The cuts are cut at the free end of the outer tube thereby weakening the same, and the engagement between the cuts and the shoulders is spaced from the free end of the inner tube. This prior-art tube connection has basically the same disadvantages as the tube connection of EP-A-797,038.

Hence, there is need for a new type of tube connection for ventilation duct systems.

### SUMMARY OF THE INVENTION

An object of the present invention is to provide an improved technique for connecting tube elements in a ventilation duct system.

This and other objects of the invention, which will appear from the following description, have now been achieved by an arrangement, a method, use and ventilation components as defined in the appended independent claims. Preferred embodiments and variants of the invention are defined in the appended subclaims.

In the arrangement of the invention, the inner tube element is provided with first coupling means for engaging corresponding second coupling means on the inside of the outer tube element, wherein at least one of the first and second coupling means is continuous about substantially the entire circumference of the associated tube element. Thereby, a quick and easy connection of the tube elements can be achieved. No extra tools or separate parts are required, and the fitter does not have to bring complicated cuts into specific positions for connection.

Furthermore, the first coupling means is provided at a distance from a free end of the inner tube element. In this section of the inner element, circumferential abutments or shoulders are easy to form in the tube wall which is an advantage. This also enhances the close fit with efficient sealing effect.

Preferably, the first coupling means is continuous about the circumference of the inner tube element. Such a coupling means is easy to form on a tube element and thereby advantageous with respect to effective manufacturing. Furthermore, it is easy to establish engagement with the matching coupling means of the outer tube element, irrespective of the shape of the matching coupling means. Thanks to the continuous first coupling means the inner tube element can be rotated to any desired angular position without jeopardizing the engagement and the connection.

In a preferred embodiment, the distance between the engaged coupling means and the free end of the inner tube element is about 7-38% of the nominal diameter of the inner tube element, preferably about 10-35% and most preferred about 14-30%. This dimensional ratios provide a close fit and a secure connection.

Preferably, the second coupling means of the outer tube element comprises depressions formed in the tube wall and angularly spaced with respect to the circumference of the outer tube element. During insertion, these depressions ride over the end portion of the inner tube element and thereby deform the tube wall of the outer tube element to a non-circular cross-section. This deformation in the engagement between the depressions and outer tube wall enhances smooth insertion and secure snap connection of the tube elements.

In an embodiment, the inner tube coupling means has at least one first abutment which is substantially perpendicular to the direction of insertion, and the outer tube coupling means has at least one second abutment which is substantially perpendicular to the direction of insertion. Such co-operating abutments provide a secure connection which prevents the inner tube element from being pulled out of the outer tube element after connection and vice versa.

In one embodiment, the coupling means of the outer tube element comprises a number of abutment edges which are angularly spaced with respect to the circumference of the outer tube element. Alternatively, a continuous recess is formed around the circumference of the outer tube element. Abutments of this kind are easy to provide in manufacture which is an advantage.

Preferably, the inner tube element comprises a circumferential groove in which is mounted a circumferential sealing ring for sealing against the inside of the outer tube element. This enables a favourable sealing of the joint between the two tube elements is secured.

The tube connection method of the invention comprises the steps of partially inserting the inner tube element into the outer tube element to a close fit, and establishing engagement between coupling means of the inner and outer tube elements, respectively, for providing connection of the tube elements, said engagement being at a distance from a free end of the inner tube element.

Further advantages of the invention and its embodiments will appear in the following description.

### BRIEF DESCRIPTION OF THE DRAWINGS

The invention will now be described further with reference to the accompanying drawings which show non-limiting embodiments and variants.

FIGS. 1-2 show—in sectional views—a tube connection according to a first embodiment of the invention.

FIG. 3 shows a tube connection according to a second embodiment of the invention.

FIG. 4 shows a tube connection according to a third embodiment of the invention.

FIG. 5 shows a tube connection according to a fourth embodiment of the invention.

FIG. 6 shows a tube connection according to a fifth embodiment of the invention.

FIG. 7 shows a tube connection according to a sixth embodiment of the invention.

FIG. 8 shows a tube connection according to a seventh embodiment of the invention with duall coupling means.

FIG. 9 shows a variant of the embodiment of FIG. 8.

### DESCRIPTION OF EMBODIMENTS

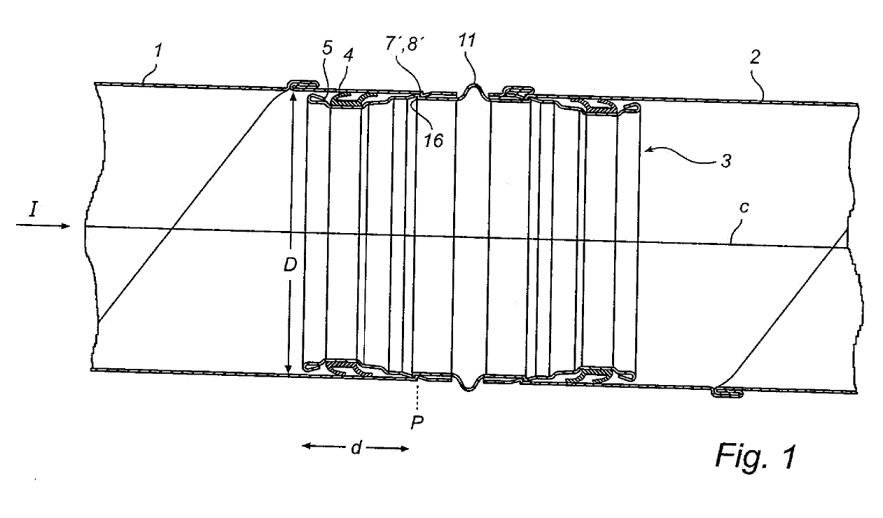

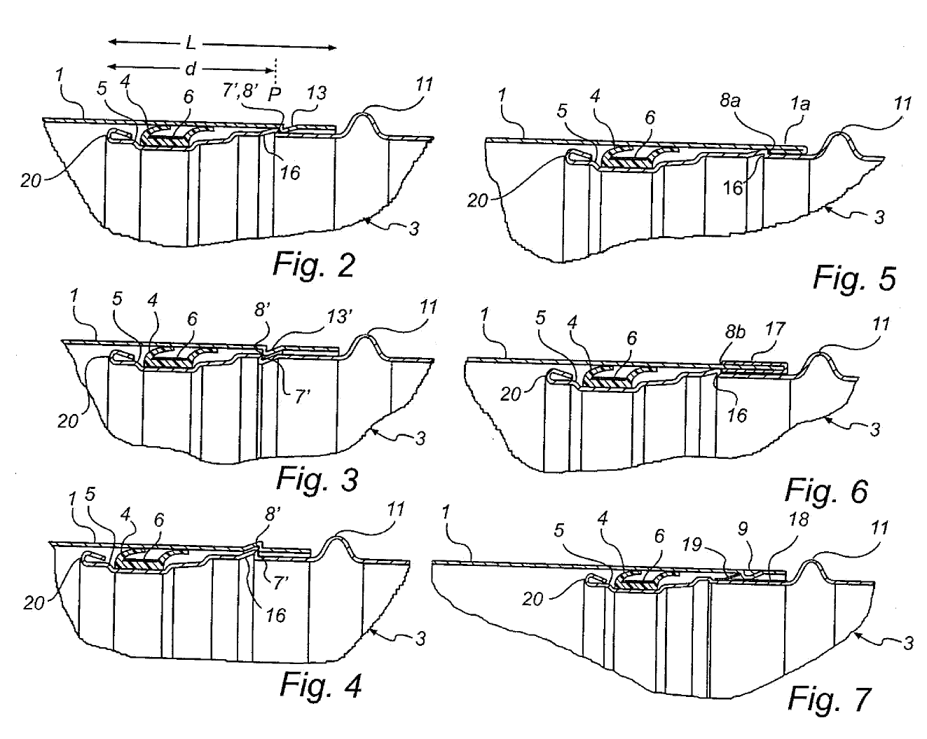

With reference to FIGS. 1-2, a basic version of this invention is described. FIG. 1 shows how a tube element or tube 1 is connected to another tube element or tube 2 by means of an intermediate tube element 3 in the shape of a connector member. The tubes 1, 2 are circular ventilation ducts included in a ventilation duct system, and the tube element 3 is in this example a so-called connector of sheet metal (for instance of the type “NPU” marketed by the applicant Lindab AB). Preferably, the tube elements 1, 2 are helically-wound lock-seam tubes of sheet metal having a wall thickness from about 0.5 mm to about 1.25 mm depending on the diameter (D) of the tubes. The inventive concept is in particular applicable to ventilation ducts within the diameter range 80-315 mm, but it can also be used for much larger diameters like 1 600 mm.

In each end portion, the connector 3 has a two-lip sealing ring 4 of rubber which preferably is of the type “LindabSafe®” marketed by the applicant Lindab AB. The sealing ring 4 is mounted in a circumferential groove 5 of the connector 3 and tightened by a circumferential strap 6.

As is shown in FIG. 1, the tubes 1, 2 are connected by the connector 3. Hence, a connection or joint is provided at each overlapping interface between the connector 3 and the tubes 1, 2. The inner tube element (the connector 3) is partially inserted with a close fit into the outer tubes 1, 2.

For obtaining a secure connection between the connector 3 and each tube, the tubes 1, 2 and the connector 3 are provided with matching coupling means for mutual engagement.

Thus, the connector 3 has an abutment 7′ with an abutment surface or edge which is substantially perpendicular to the axial direction of insertion and to the centre axis C of connector 3. In this embodiment, the abutment 7′ is continuous about the entire circumference of the connector 3 but variants are feasible (to be described below). The abutment 7′ forms the coupling means of the connector 3.

The outer tube 1 has at least one abutment 8′ formed in the tube wall for engagement with the abutment 7′ of the connector 3. In an embodiment, the abutment 8′ is formed by a number of spaced depressions or recesses 9 in the tube wall about the circumference of the tube 1 (see FIG. 7). The recesses 9 do not go through the tube wall, so there is no risk of air leakage. The abutment 8′ has an abutment surface or edge which is substantially perpendicular to the axial direction of insertion and to the centre axis C of the tube 1. The abutment 8′ forms the coupling means of the outer tube 1.

For connection, the connector 3 is inserted into the tube 1 and the mutual coupling and engagement means 7′ and 8′ are operable to secure the connection. As can be seen in FIGS. 2-4, the coupling means of the outer tube 1 has been moved past the coupling means of the connector 3 and a connection has been established after insertion. The coupling means are interlocked in a locked position. The abutments 7′, 8′ are snapped to this position and establish the locking structure and function. For facilitating the engagement, the abutments 7′, 8′ have ramp surfaces.

An outer circumferential bead 11 of the connector 3 provides an external abutment or stop for the free ends of the two tubes 1, 2 to be connected with the connector 3. Hence, the bead 11 determines the partial insertion of the connector 3 in the tubes 1 and 2 (see FIG. 1).

The mutual engagement between the abutments 7′ and 8′ is established in a plane P which is perpendicular to the direction of insertion.

In FIGS. 2-3, the coupling means of the outer tube 1 is formed by a continuous circumferential groove or recess 13, 13′ about the entire circumference of the outer tube 1.

The abutment 7′ of the inner tube or connector 3 does not have to be entirely continuous, but it can also comprise abutments edge portions which form arcs of a circle (not shown) and which are distributed about the circumference of the connector 3.

As shown in FIGS. 2 and 4-6, the abutment of the inner tube or connector 3 is formed by a continuous circumferential shoulder 16 extending radially outwards from the outside of the connector wall adjacent to the groove 5 in which the sealing ring 4 is mounted. These embodiments have the advantage that the abutment shoulder 16 is rather easy to produce in standard production equipment.

A modified sealing ring can be used a base portion of which is clamped by the bent free end 20′ of the connector 3 (not shown). This sealing ring can be of the type described in the pamphlet “SPIRO® System” issued by Spiro International S.A. (1999).

In all embodiments, the coupling engagement is disposed at a distance (d) from the free end of the connector 3, that is “inside” the sealing ring 4 with respect to the tubular body of the connector 3. Practical tests have shown that there are favourable ratios between said distance (d) and the nominal diameter (D) of the inner tube or connector 3. Secure and reliable tube connections are achieved if this ratio (d/D) is within the approximate range 7-38%, preferably about 10-35% and most preferred about 14-30%. Another relevant parameter is the ratio between said distance (d) and the insertion length (L), see FIG. 2. Preferably, this ratio (d/L) should be within the approximate range 50-85%, preferably about 70-80% depending on the diameter of the tubes. If this ratio between the distance (d) and the insertion length (L) is used, a favourable snap locking effect is achieved and a very reliable tube connection.

The embodiments of FIGS. 2-9 are based on engagement between abutment surfaces or edges 7′ and 8′ associated with continuous circumferential grooves 13 or shoulders 16, respectively, or spaced depressions 9.

In the embodiment of FIG. 5, the free end 1 a of the outer tube 1 is bent inwardly for forming an abutment 8a to be engaged with the shoulder 16 of the connector 3. Alternatively, as is shown in FIG. 6, a separate member 17 (for instance of sheet metal) is fastened at the free end of the outer tube 1 and an abutment 8b is formed in a similar manner.

Still another embodiment shown in FIG. 7 has a circumferential strap 18 (for instance of resilient sheet metal) clamped about the connector 3. The strap 18 has an inclined circumferential and continuous abutment portion 19 for engagement with the recess 9 of the outer tube 1.

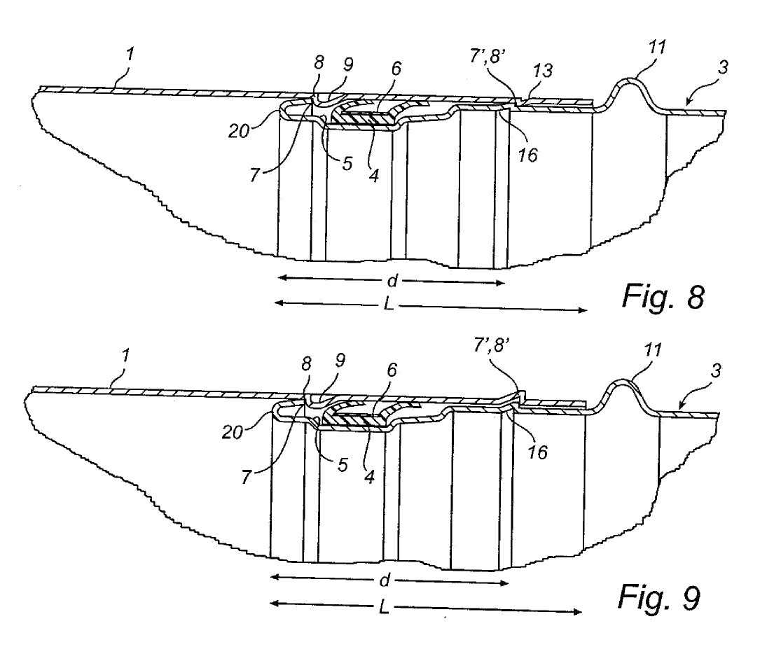

An embodiment with two axially spaced coupling portions is illustrated in FIGS. 8-9. A first coupling in the end portion of the connector 3 is established by the engagement between an abutment 7 of the bent free end 20 of the connector 3 and a matching abutment 8 formed by spaced depressions 9 of the outer tube 1. A second coupling is established by the coupling means 7′, 16 which are formed on the outside of the connector 3 and which match the corresponding coupling means 8′, 13 formed on the inside of the outer tube 1.

The connector coupling means 7′, 16 are provided at a distance (d) from the free end 20 of the connector 3. Preferably, either of these coupling means 7′, 16 or 8′, 13 (respectively) comprise spaced depressions which cooperate with a continuous abutment forming the matching coupling means. This embodiment is in particular suitable for large-diameter ventilation ducts which may “flex” in the overlapping connection. The combined coupling effect in two axially spaced engagement portions provides a secure tube connection which reduces the “flexing” in the joint between the tubes.

Although this description only has discussed tube elements of circular cross section, it should be mentioned that the inventive concept is applicable also to tubes and ventilation ducts of different cross sections, such as flat-oval, rectangular, etc. The coupling means of the tubes are designed in a corresponding manner. A common feature of all embodiments of the invention is that the tube coupling means and abutments are configured in such a way that no open cuts or apertures are left in the tube walls which could lead to air leakage.

It should be emphasised that the inventive concept is not limited to the embodiments described here, and that modifications are feasible without departing from the scope of the invention defined in the appended claims. For instance, the number of abutment edges can vary. Furthermore, the inner tube can be other than a connector, for instance a bend, a T-piece, a silencer, etc.

1. An arrangement for connecting tube elements of a ventilation duct system, comprising an inner tube element (3) which is configured to be partially and with a close fit inserted into and connected with an outer tube element (1), wherein the inner tube element (3) comprises external first coupling means (7′, 16; 19) for engagement with corresponding second coupling means (8′; 8a; 8b; 9) provided on the inside of the outer tube element (1), the engagement between said first and second coupling means providing said connection of the tube elements (1, 3) after insertion, and said engagement occuring occurring in a plane (P) substantially perpendicular to the direction of insertion, wherein at least one of said first and second coupling means extends along substantially the entire circumference of the associated tube element and wherein said first coupling means is provided at a distance (d) from a free end of the inner tube element (3).

2. An arrangement as claimed in claim 1, wherein said first coupling means (7′, 16; 19) is continuous along the circumference of the inner tube element (3).

3. An arrangement as claimed in claim 1, wherein said distance (d) from said free end of the inner tube element (3) is about 7-38% of the nominal diameter (D) of the inner tube element (3).

4. An arrangement as claimed in claim 3, wherein the ratio (d/D) between said distance (d) and said nominal diameter (D) is about 10-35%, preferably about 14-30%.

5. An arrangement as claimed in claim 2, wherein said second coupling means (8) of the outer tube element (1) comprises depressions (9) formed in the tube wall and angularly spaced with respect to the circumference of the outer tube element (1).

6. An arrangement as claimed in claim 1, wherein said first coupling means comprises at least one first abutment (7′, 16) which is substantially perpendicular to the direction of insertion and wherein said second coupling means comprises at least one second abutment (8′; 8a; 8b) which is substantially perpendicular to the direction of insertion (I), said abutments co-operating for securing said connection.

7. An arrangement as claimed in claim 6, wherein said second coupling means (8) comprises a plurality of second abutment edges which are angularly spaced with respect to the circumference of the outer tube element (1).

8. An arrangement as claimed in claim 6, wherein said outer tube element (1) comprises a circumferential recess (13, 13′) defining the second abutment (8′) which is continuous along the circumference of the outer tube element (1).

9. An arrangement as claimed in claim 6, wherein said first abutment (7′) is formed on a circumferential shoulder (16) provided on the outside of the inner tube element (3) at a distance (d) from the free end thereof.

10. An arrangement as claimed in claim 1, wherein the inner tube element (3) comprises a circumferential groove (5) in which is mounted a circumferential sealing ring (4) for sealing against the inside of the outer tube element (1).

11. An arrangement as claimed in claim 10, wherein the sealing ring is mounted in the groove (5) by clamping a bent end of the inner tube element (3) on a portion of the sealing ring.

12. An arrangement as claimed in claim 1, wherein the inner tube element (3) comprises means (11) configured to be engaged by the free end of the outer tube element (1) for determining said partial insertion.

13. An arrangement as claimed in claim 1, wherein the inner tube element (3) comprises further outer coupling means (7) in an end portion of the inner tube element (3), said further coupling means (7) being configured to engage with further inner coupling means (8) of the outer tube element (1).

14. An arrangement as claimed in claim 1, wherein the outer tube element is a helically-wound lock-seam tube (1) of sheet metal, and wherein the inner tube element (3) is of sheet metal as well.

15. An arrangement as claimed in claim 14, wherein the inner tube element is a connector (3).

16. A ventilation fitting to be used as an inner tube element in the arrangement as claimed in claim 1, wherein the ventilation fitting comprises said first coupling means.

17. A ventilation duct to be used as an outer tube element in the arrangement as claimed in claim 1, wherein the ventilation duct comprises said second coupling means.

18. A ventilation duct system, comprising at least one tube connection arrangement as claimed in claim 1.

19. Use of an arrangement as claimed in claim 1 for connecting tube elements, ducts and/or fittings included in a ventilation duct system.

20. A method for connecting tube elements of a ventilation duct system by a tube connecting arrangement as claimed in claim 1, comprising the steps of:
a) partially inserting an inner tube element into an outer tube element to a close fit, an
b) establishing engagement between coupling means of the inner and outer tube elements, respectively, for providing said connection of the tube elements.
